(19) United States
(12) Patent Application Publication
(10) Pub. No.: US 2006/0292215 A1
(43) Pub. Date: Dec. 28, 2006

(54) Abscisic acid against cancer

(76) Inventor: Gonzalo Romero M, Saint Petersburg, FL (US)

Correspondence Address: GONZALO ROMERO M, 455 39TH AVE NORTH APT #2, SAINT PETERSBURG, FL, 33703-6118, US

(21) Appl. No.: 11/472,128

(22) Filed: Jun. 21, 2006

Related U.S. Application Data
(60) Provisional application No. 60/692,617, filed on Jun. 22, 2005.

(51) Int. Cl. A61K 9/48; A61K 31/19

(52) U.S. Cl. 424/451; 514/557

(57) ABSTRACT

Abscisic acid (ABA) a naturally ocurring plant hormone has been identified in this invention with potent properties to fight cancer. ABA is able to produce a hyperpolarization condition on plasma membrane through a decrease of intracellular Na⁺ and K⁺. Such phenomenon is produced in cancer cells by mediation of ion channel and activation of the signalling G-protein pathway. ABA aborting sustained depolarization in malignant tissue will produce a change in the configurational state of cell from a damage to a normal state. Additionally, a positive polarization of HCG outer layer accomplished through a removal of electrons will permit immune system cells coming close to cancer cells for destruction.

### CROSS-REFERENCE TO RELATED APPLICATIONS

This application is entitled to the benefit of provisional Patent Application Ser. No. 60/692,617 filed Jun. 22, 2005. Otherwise, omit this section.

### BACKGROUND of the INVENTION

1. Field of the Invention

This invention has been found in the field of medical treatments of drugs biologically affecting the human body and related to counteracting different types of cancer, which have potentially unlimited growth and expand locally by invasion and systemically by metastasis.

2. Description of the Prior Arts

Abscisic acid (ABA) is a natural occurring plant hormone also denominated Abscisin II or Dormin. It is chemically named as [S-(Z,E)]-5-(1-Hidroxy-2,6,6-trimethyl-4-oxo-2-cyclohexen-1-yl)-3-methyl-2,4-pentadienoic acid (Merck Index 1996, p. 2). ABA molecular structure is showed in FIG. 1.

ABA structure is a 15-C sesquiterpene synthesized in chloroplasts and likely also under extrachloroplasts biosynthetic pathway. ABA is a weak acid (C₁₅H₂₀O₄) soluble in many organic solvents. Addicott et al (1969), Milborrow (1974), and Walton (1980) have studied physical and chemical characteristics of ABA.

The natural compound has been indicated as (S) or (+), its synthetic or racemic substance as (RS) or (+/−), and its enantiomer as (−).

ABA natural form (+), synthetics or enantiomers, related and derivatives, all of these are subjects of the present invention.

Some of the ABA related and derivatives mentioned here are: 2-trans-abscisic acid; Phaseic acid; 2-trans-phaseic acid; (+)-abscisyl-beta-D-glucopyranoside; (R)-abscisic acid; 2,4-trans, cis-abscisic acid. Addicot et al (1969), p. 144, has all those substances pictured. In addition, Milborrow (1974), pp. 261-272, has defined the following chemical structures: 1′,4′-cis-diol of (+)-abscisic acid; 1′,4′-trans-diol of (+)-abscisic acid; (+)-xantoxin (2-cis); (+)-xanthoxin (2-trans); (+)-xanthoxin acid (2-cis); xanthoxin acid methyl ester (2-cis-isomer); (+/−) abscisic alcohol; (+/−)-abscisic aldehyde. Also Milborrow (1974) has characterized the following forms: XIII, XIV, XV, XVI, XVII, XVIII, XIX, XX, XXI, XXII, XXIII, XXIV, XXV, XXVI, XXVII, XXVIII, XXIX, XXX, XXXI, XXXII, XXXIII, XXXIV, XXXV, XXXVI, XXXVII, XXXVIII, XXXIX, XL, XLI, XLII, XLIII, XLIV, XLV, XLVI, XLVII, XLVIII, XLIV, XLV, XLVI, XLVII, XLVIII, XLIX, L, LI, LII, LIII, LIV, LV, LVI, LVII, LVIII, LIX, LX, LXI, LXII, LXIII, LXIV, LXV. Many of those mentioned substances have been found by researchers generating a variety of effects in plants.

ABA as a potent inhibitor has been studied through an extensive bibliographical reference in biochemical and physiological mechanisms such as: water stress and stomatal control, dormancy, abscission, senescence, growth ihibition, permeability and hydraulic conductivity of plasma membrane.

Dormancy has been mentioned by Walton (1980), p. 475, mostly correlated with maintenance of it than by induction. Abscission which is a lysigenous breakdown of cells accelerated by a rapid development of pectinases, cellulases, and proteases has been suggested by Borman et al 1967, p. 125, and by Addicot et al 1969, p. 156. Senescence, identified by multiple effects such as fruit maturation, aging and plant death has been observed by Milborrow 1974, p. 293. In addition, Glinka et al (1971, 1972) and Windsor et al (1992), p. 59 have mentioned an increase of membrane permeabilty caused by ABA.

Growth Inhibition

Growth inhibition in plants as a property of ABA is a phenomenon related with cancer and is of mayor concern for mentioning here.

ABA inhibited oat mesocotyl growth by increasing ABA or dormin concentrations, whatever levels of indol acetic acid (IAA) or gibberellins (GA) were given (Milborrow, 1966, p. 154). ABA also inhibits tomatoes lateral buds outgrowth (Tucker 1977). Probable changes in tissue growth by ABA effects might be provoked in nucleic acids and metabolism of proteins. Dekock et al 1978, p. 508, suggests that ABA exerts its primary response at the nucleic acid level. It specifically affects translation or transcription of protein synthesis.

Stomatal Movements

ABA acts in many physiological processes in plants, especially during stomatal closure in conditions of limited water supply. Stomatal closure involves loss of ions and reduction of osmotic pressure (Mansfield et al 1971, p. 147; Dekock et al 1978, p. 506; MacRobbie, 1997, p. 515). Loss or entry of anions and cations is mediated by signalling activation of ion channel (Schroeder et al 2001, p. 328; MacRobbie, 1997, p. 515).

Apparently ABA in the apoplast must be inhibited or moved during plant rewatering, which starts to open stomata. Mechanisms of inhibition probably comprise: relocation of the hormone in other plant compartments outside the stomatal complex, ABA uptake into cytoplasm to be metabolized or stored, and inhibition of synthesis.

In normal conditions, stomatal opening and closure respond to light, without exogenous application of ABA. X-ray images in tobacco leaves demonstrate a K⁺ increase in guard cells as stomata open in the light, and a decrease as stomata close in the dark (Sawhney et al 1969, pp. 1351-1353; Humble et al 1971, pp. 448-450).

The pH changes in stomas generated by light have been thought to occur in relation to photosynthesis. A reduction of CO₂ concentration in guard cells as a result of photosynthesis consumption, causes a rise in pH. During the darkness, photosynthesis stops, and CO₂ concentration rises as a result of respiration (Devlin 1966), p. 72. The latter phenomenon produces a decrease in pH. Mesophyll and epidermis cells of leaves also intervene in the stomata response. They are able to store ABA when intracellular pH is relatively high during the day, but at night when photosynthesis stops and intracellular pH decreases, ABA is released outside plasma membrane activating stomatal closure. This daily cycle and pH changes can be understood through the ion trapping concept of, ABA.

ABA is a weak acid, which preferentially acumulates in the more alkaline compartments of the leaf. At an acidic pH (5.2 TO 6.5) more ABA will be present in its lipophilic undissociated form (ABAH). Such form can diffuse across the plasma membrane into the more alkaline compartments of the cytoplasm. In such compartments of the mesophyll and epidermis cells which have a pH between 7.2 and 7.4, it dissociates to lipophobic form (ABA and H⁺), which becomes trapped inside the cell (Wilkinson et al 1997, p. 559).

During normal stomatal opening, uptake of K⁺ is mediated by K⁺ ion pump (Sawhney et al 1969, p. 1350). Outlaw (1983) in his review current concepts on the role of potassium in stomatal movements remarks in the abastract, p. 302, that K⁺ uptake by plant cells is mediated by an ATPase that pumps protons across the plasmalema. Protons released during stomatal opening increase medium acidification and they are roughly equivalent to the amount of K⁺ reportedly taken. This reveals an exchange of H⁺/K⁺ across the plasmalema. Cytoplasmic K⁺ has a bigger concentration than K⁺ in the apoplast, thus it seems obvious a use of energy by cells for transporting ions as K⁺ against a gradient of concentration.

Loss of Solutes by ABA Effect

Mansfield et al (1971), p. 147, Showed In Histochemical Tests, that K⁺ concentration in guard cells of C. communis was reduced by ABA treatment, while the starch content of chloroplasts increased.

ABA also moves other cations as Ca²⁺ to be transported inward (MacRobbie 1997, p. 515; Schroeder 2001, p. 328), and anions to be transported outward (Schroeder 2001, p. 328).

Dekock et al (1978), p. 506, working with Lemna gibba fronds and ABA, detected a marked intracellular decrease in K⁺ and Na⁺, an increase in Ca, Mg, Fe, and insoluble P and a marked decrease in P/Fe and K/Ca ratios.

MacRobbie (1997), page 515, also confirms and gives a better explanation of the phenomenon. ABA closes stomatal pores by inducing net loss of K⁺ salts (including rubidium) from guard cells, involving net efflux of both anions and cations from the vacuole to the cytoplasm, as well as from the cytoplasm to the outside. Transport of ions through the plasma membrane is produced by the activation of the ion channel and ion pump.

Humble et al (1971), p. 451, under research of stomatal movements determined that K⁺ is the specific ion involved, not showing significant importance, the rest of the ions. It takes a real relevance, due to that ABA also moves other cations as Ca²⁺, to be transported inward and anions to be transported outward.

ABA also inhibits K⁺ uptake which is required for stomatal opening (Schroeder et al 2001, p. 328). ABA in roots has a similar efect as it is exerted in leaves during stomatal opening. In excised barley roots of Hordeum distichon, ABA inhibits accumulation, transport and uptake of K⁺ and Na⁺ to avoid osmotic stress during drought (Behl et al 1979, p. 335). Here, it is also possible to observe a definitive influence of ABA over cation Na⁺. Wilmer et al (1969) also demonstrated that Na⁺ and K⁺ are important in stomatal mechanisms.

In normal cells an action of ABA will be exerted mainly over K⁺ due to this cation is more important in the cytoplasm. Nevertheless, ABA action in cancer cells will be exerted over Na⁺, because such cation predominates in this type of cell.

Positive Polarization and Hyperpolarization Effect in Plasma Membrane

Tanada (1971), p. 461, in his study Anatagonism between indoleacetic acid and abscisic acid on a rapid phytochrome-mediated process concluded that, phytochrome acts in conjunction with hormones such as IAA and/or ABA to bring fast changes in surface charges. Tanada suggested these hormones have opposing effects on the surface potentials: ABA inducing a positive and IAA inducing a negative potential. ABA synthesis location has been recognized in chloroplasts mediated by phytochromes absorbing light, which increases a concentration of ABA. Tanada's experiment demonstrated that red photons could increase concentration of ABA relative to that of IAA thereby bringing about a positive membrane potential which causes adhesion of root tips to a negatively-charged glass suface.

Hartung et al (1980), pp. 257-258, recorded a hyperpolarization effect of ABA in Lemna gibba G1 on a membrane electrochemical potential difference (EPD). Treatment with 10-100 mcM ABA produced a potential increase in average of 85 mV. This research mentions that a decreased intracellular K⁺ concentration could generate membrane hyperpolarization of Lemna cells. After 15 hours ABA treatment, EPD increased from −109 mV to −194 mV. K⁺ outside of the cytoplasm causes a positive charge on plasma membrane and it also increases charge difference between the cytoplasm and surface membrane.

Osterhout on 1933, p. 126, analyzed electrical behavior of K⁺ in large plant cells saying that, if there is an outwarly directed potential difference of 100 mV or more it will give a corresponding current of injury. He also mentioned that, cell non-aqueous layers have 1 micron in thickness and a potential drop of 100 mV or more across 1 micron, is equivalent to 100 volts across a layer 1 millimeter in thickness.

EPD results, from a separation of positive and negative charges across the cell membrane. This separation of predominantly positive charges outside and negative charges inside the membrane at rest is maintained because the lipid bilayer acts as a barrier to the diffusion of ions. It gives rise to an electrical pd and the resting potential can be disturbed whenever there is a net flux of ions into or out of the cell. A reduction of the charge separation is called depolarization and an increase in charge separation is called hyperpolarization. In normal cells, a higher concentration of anions relative to K⁺ mainly produces a net negative charge inside of cells. Net positive charges outside of cells is produced by a bigger concentration of Na⁺ relative to CI⁻.

The ABA hyperpolarization phenomenon mentioned by Hartung et al (1980) is distinct to ABA positive polarization effect researched by Tanada (1971). The latter effect is a subtle phenomenon related intimately to the first one, which it will be disclose widely ahead in this invention.

ABA in Relation to a Physiological Relationship with Mammalians

Le Page-Degivry et al (1986), pp. 1155-1156, reported a presence of ABA in the central nervous system of pigs and rats. Identification of ABA by using a radioimmunoassay in different tissues demonstrated a bigger amount of ABA found in brains than any of the other tissues. The final product of purification had the same properties as ABA, inhibiting stomatal aperture of abaxial epidermis strips of Setcreasea Purpurea Boom (Commelinaceae). They remarked that presence of ABA can not be considered a consequence of a diet containing ABA, and suggested identification of metabolic pathways for ABA biosynthesis in the brain.

ABA is a plant hormone sharing certain physiological mechanisms also observed in animals and humans. Coursol et al (2003), p. 651, in the research Sphingolipid signalling in Arabidopsis guard cells involves heterotrimeric G proteins, showed that a metabolite denominated sphingosine-1-phosphate (SIP) functions in animals as an intracellular messenger and an extracellular ligand for G-proteins-coupled receptors of the receptor family, regulating diverse biological processes. In This research it was discovered in plants that SIP is a signalling molecule involved in ABA regulation of guard cell turgor. It also was reported that an enzyme responsible for SIP production, sphingosine kinase (SphK), is activated by ABA in Arabidopsis thaliana, and is involved in both inhibition of stomatal opening and promotion of stomatal closure. Signalling G-proteins pathway is the responsible mechanism stimulating ion channel activity in stomatal movements.

Plant Hormone Effects in Humans

Naturally ocurring cytokinins such as kinetin and zeatin, which intervene in plant cellular division have been promoted and patented in Europe and USA for treatment of human skin aging by an international biopharmaceutical corporation denominated senetek. These patents have showed and proved that, kinetin is capable of delaying or preventing a host of age-related changes of human skin fibroblasts grown in laboratory culture.

Fibroblasts are believed to be at the center of age-related changes of the skin. These cells produce collagen and elastin, the two proteins most clearly tied to the development of wrinkles, sagging and laxity of the skin. Also IAA has been promoted in USA for human skin aging and recognized as the principal promoter of cellular division in plants. Additionally GA has been structurally found as molecules roughly analogous to the steroid group of animal hormones. Steroids have an enhancing effect in human cells and it has been used in sport activities for increasing musculature size. In plants, GA produces cellular elongation, which is a similar enhancing effect induced by steroids in humans.

ABA in Relation to the Disease of Cancer

This invention was initially started by considering that, plant hormones such as IAA, GA and cytokinins stimulate cellular growth in plants. Conversely, ABA manifests an antagonist effect in plants by producing cellular growth inhibition. Aforesaid experiments of Tanada (1971) show an opposite effect of ABA and IAA in plasma membrane polarization. As it will be explained later in this invention, EPD has powerful influence in cells, stimulating up a quiescent stage or activating cellular mitosis.

ABA was mentioned for the first time in relation to cancer by Dr. Virginia Livingston in her U.S. Pat. No. 3,958,025 (1976) denominated, abscisic acid tablets and process. In this invention she experimented with ABA in mice exppressing properties of ABA of neutralizing a microbic chorionic gonadotropin which was similar to Human Chorionic Gonadotropin (HCG).

Such statement defined by Dr. Livingston was essentially a complementary or collateral research beside her central focus of investigation, which was about a type of tumor-associated bacteria produced by Progenitor cryptocides expressing HCG and developing cancer. Such findings reported back in the early 1970's were erroneously dismissed. After Dr. Livingston's research, it was recognized that cancer cells and certain types of tumor-associated bacteria also produce this hormone and cancer, as indicated by her.

Some coagulase-negative Sataphylococcus species isolated from cancer patients expressed HCG, but not every isolated bacterial strain did it, Acevedo (2002), p. 860. Such studies confirmed that, Progenitor cryptocides is not the unique bacterium expressing HCG as postulated by Livingston and associates, but her conclusions and investigations were right. Dr. Livingston's findings were a first indication of a possible synthesis of a mammalian protein hormone by a microbial organism, Cohen et al (1976), page 410.

Likewise as it ocurred with the central focus of Dr. Livingston's investigation, it also took place with her complementary or collateral research about ABA and its capacity of neutralizing HCG. The central topic of Dr. Livingston research was verified aftermath, but not her complementary or collateral research, which it is motive and impulse of this invention requiring it a confirmation.

In 1984 Dr. Livingston published her book The Conquest of Cancer, in which she mentioned results on pp. 15-38, of applying in humans an integral treatment stimulating the immune system and administering ABA by a dietetic via.

Since Dr. Livingston's works, which were crystalized in researches, books and inventions, nobody else has studied and mentioned ABA in relation to cancer. Instead of, ABA has been studied around the world in connection to agricultural concerns, specifically about drought and water stresses.

This invention may be considered a forward step of U.S. Pat. No. 3,958,025 (1976), claiming and clarifying ABA mechanisms and properties against cancer.

### OBJECTIVES of the INVENTION

1. Stimulate and encourage researchers to keep investigations going of Dr. Virginia Livingston about ABA and its properties against cancer.

2. Define probable mechanisms of ABA to fight cancer.

3. Produce technical information that can be useful to make a clinical research and for manufacturing an adequate pharmaceutical medicine.

4. verify testimony expressed by Dr. Livingston about ABA capacity to neutralize the hormone of cancer.

### DRAWINGS

Following drawings are presented in this invention: 1. Molecular structure of ABA. 2. Molecular structure of sialic acids (SIA). 3. Curve of mechanism of Na⁺ in normal proliferating cells. 4. Concentration gradient of Na⁺ Toward the extracellular fluid. 5. Concentration gradient of Na⁺ toward the cell. 6. Electroscope in contact with a negatively charged body. 7. Electroscope in contact with a positively charged body. 8. Curve of ABA uptake, 9. Curve of a buffer solution.

### REFERENCE NUMBERS

10 Electrochemical Potential Difference (EPD) with values between 0 and −70 mV.
12 sodium concentrations in percent.
14 isoelectric point.
16 curve of intracellular sodium concentration [Na⁺] i.
18 curve of extracellular sodium concentration [Na⁺] e.
20 quiescent stage.
22 mitogenesis.
24 hyperpolarization.
26 depolarization.
28 first layer (stern layer).
30 cancer cells plasma membrane showing negative charges.
32 cations attached to plasma membrane.
34 second layer (diffuse layer).
36 positive and negative ions in diffuse layer.
38 third layer (HCG layer).
40 negative charges of SIA.
42 repulsed or attracted cations in third layer.
44 metal ball of the electroscope.
46 metal rod of the electroscope.
48 metal leaves of the electroscope.
50 isolating material or gasket.
52 glass container of the electroscope.
54 negative or positively charged body or ion.
56 electrons.
58 electron transfer.
60 curve of ABA uptake.
62 ABA uptake concentrations in percents.
64 values of pH between 3 and 7 in a medium outside plasma membrane.
66 area of ABA uptake maximum efficiency during stomatal closure.
68 area of ABA uptake minimum efficiency during stomatal closure.
70 pK of ABA (4.7).
72 values of pH between 4 and 7.
74 Relative CONCENTRATIONS of carbonic acid (H₂CO₃) ranging between 0 and 100%.
76 Relative concentrations of HCO₃⁻ (bicarbonate) ranging between 0 and 100%.
78 pK of carbonic acid (6.1).
80 curve of buffer solution.
82 region of maximum buffering capacity.
84 normal blood pH.

### DETAILED DESCRIPTION OF THE INVENTION

ABA in Relation to Human Chorionic Gonadotropin

Dr. Livingston postulated in her U.S. Pat. No. 3,958,025 (1976), col 8, line 50, that a hormone immuologically identical to HCG denominated microbic chorionic gonadotropin, produced from progenitor cryptocides, might be opposed or neutralized by a growth retardant in vitro. Such a growth inhibitor was identified as ABA.

Experiments in vivo of Dr. Livingston, mentioned in her patent (col 9), demonstrated capacity of ABA to neutralize HCG. For determination of cancer survival rate, she used C57BL/6J mice and C1498 transplated tumor with myeloid leukemia purchased from the Jackson laboratory in Maine. This type of cancer was lethal in mice in 10-15 days. ABA furnished by Hoffmann-Laroche was suspended in saline and administered as suspension. Groups of 10 mice were used for treatment for a total of 7 days.

The following rate of survival was noted at the end of 14 days: Group I: control saline intraperitoneal (i.p)=3 survivors; Group II ABA 1 mg/kg (i.p)=9 survivors; Group III ABA 10 mg/kg (i.p)=10 survivors; Group IV ABA 10 mg/kg (oral)=6 survivors; Group V ABA 100 mg/kg (oral)=9 survivors.

It was concluded that ABA has a marked effect in the inhibition in mice (C57BL/6J) of the tumor system (C1498).

In 1984, Dr. Livingston obtained, pp. 15-38, after treatment of 62 random cases in humans with cancer a success rate around 82%, not considering it inconclusive cases. She applied only a digestive treatment with ABA plus an elimination of the bacterium P. crytocides with her vaccine.

Since 70 years ago, Agrobacterium tumesfaciens was identified as the cause of crown gall disease which is characterized by formation of neoplasm (galls) in plants. Beijersbergen et al (1992), p. 1324, determined that such bacterium causes the disease by a DNA transfer.

Actually it is recognized that, bacteria are not the only agents for inducing cancer; viruses, chemical compounds (toxins), physical elements such as prolonged exposition to solar radiation and other factors can provoke the disease.

Appearance of HCG in tumor cells, induced or not by a specific agent as bacterium or it ocurring in placenta or membranes of sperm cells, is a natural mechanism for protecting foreign cells against the immune system of a host organism. HCG is a sialoglycoprotein hormone produced by the human placenta having by function maintainance of the steroid hormone secretions of the corpus luteum and protecting the embryo and fetus against the immune system of the mother. A medicine against cancer must have a fundamental property of counteracting HCG to facilitate viability of tumor destruction by the immune system.

HCG is a glycoprotein containing oligosaccharides. The hormone is composed of two subunits, alfa and beta subunits, having a total of 244 aminoacids and forming a complex associated to the cellular membrane.

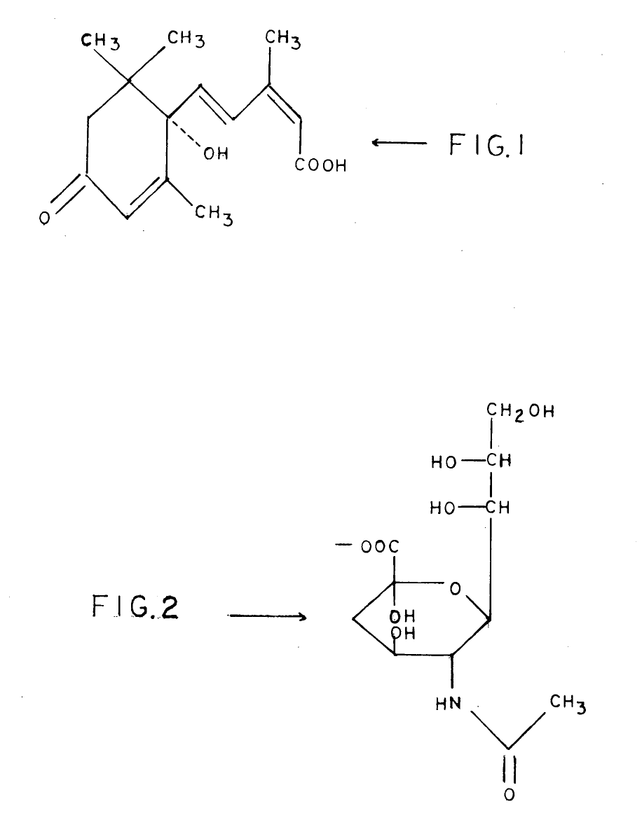

According to Acevedo (2002) pp. 135-136, alfa subunit contains two chains of N-linked oligosaccharides attached to aspargine with two molecules of n-acetyl-neuraminic acid, known as SIA (FIG. 2). The beta subunit contains. Four chains of O-linked oligosaccharides attached to the four serines of the HCG beta carboxy-terminal peptide with a total of six molecules of SIA.

The high content of SIA gives the membrane-associated HCG molecule a very high negative charge. SIA appears to be the regular components of all types of mucoproteins, mucopolysaccharides and certain mucolipids.

Cells from the human immune system express in their membranes, normal negative charges. Equal polarity of HCG and immune system cells make such cells immunologically inert and unable to get close and attack tumor cells (Acevedo 2002, p. 136). Specific inhibition of human natural killer cell by SIA and sialo-oligosaccharides has been researched by Van Rinsum et al (1986), and Published Through the International Journal of Cancer, Vol 38, pp. 915-922.

In addition to the mentioned cancer blockage created by repelling charges against immune cells, HCG also stimulates malignant growth which is summarized in the abstract 227 of Raikow et al (1987), p. 57, issued in the annual meeting of the American Association For Cancer Research.

Others researchers as Stern et al 1999, p. 367 has related increased negativity in cancer cell plasma membrane with a loss of electrons and protons toward extracellular space. This loss of electron/proton homeostasis and reversion to a glycolytic state are esteemed the basis of their proposed model of carcinogenesis. In this model, DNA abnormalities are considered contributory or secondary phenomena.

ABA In Relation to Metabolism and Transport of Cations in Cancer Cells

According to A. Keith Brewer (1984), p. 1, in his research named, The High pH Therapy for Cancer, Test on Mice and Humans, mass spectrographic and isotope studies have shown that K⁺, Rb⁺ and specially Cs⁺ are most efficiently taken up by cancer cells. Elements such as K⁺ exert important functions in cancer cell, as e.g., it transports glucose into the cell (Brewer 1984, p. 2). K⁺ is also important in actively proliferating growing tissues such as embryonic and cancer cells (Delong et al 1950, p. 721).

Despite above-mentioned statements about K⁺, cancer cells have lower K⁺ concentrations and higher Na⁺ and water content than normal cells (Cone, 1975). Likewise, Seeger et al (1990), cited in Haltiwanger (2003), p. 9, in his monograph, the electrical properties of cancer cells, sustains that cancer cells have altered their membrane composition and permeability. This results, in movement of K⁺, Mg²⁺, and Ca²⁺ out of the cell, and acumulation of Na⁺ and water inside.

Malignant transformations of cancer cells have caused a differential preference of cations in relation to the negative charges concentrated in surfaces of the intracellular protein matrix.

G. N. Ling initiated this line of investigation about mentioned above differential preference of cations in cancer cells in the 1960's (Cope, 1978, p. 466), who called it the association-induction hypothesis. Cope called such phenomenon the tissue damage syndrome, because damage in any tissue, produces a similar set of changes in salt and water content. According to Ling (1960) proteins of cells are able to exist in either of two different configurational states: a normal configuration, and a damaged configuration. In a normal state, negatively charged sites on the protein matrix have a large preference for association with K⁺ rather than Na⁺, and cell water is highly structured. The result is high cell K⁺ and low cell Na⁺. In the damaged cell, proteins lose preference for association with K⁺ rather than Na⁺, also they lose their ability to structure water, with the result K⁺ leaves the cell, and is replaced by Na⁺, and the water content of the cell increases.

On the other hand, Ca²⁺ in cancer cells is contained only at about 1% of that in a normal cell (Brewer 1984, p. 2). Likewise, Delong et al, 1950 p. 718 and Ambrose et al, 1956 p. 576 reveal that, tumor tissues show a decreased Ca²⁺ content in comparison with normal tissues.

Limited absorption of Ca²⁺ is a consequence of the anaerobic metabolism of cancer cells pointed out by Warburg (1925), p. 310. Cancer anaerobic metabolism is exclusively supported by the biochemical mechanism of glycolisis. It degrades glucose from the blood producing lactic acid and alcohol. An anaerobic cell can only produce two ATP's from the metabolism of a glucose molecule, while those aerobic cells can derive 36 ATP's from burning a glucose molecule. That minimal energy provokes: a lack of cellular division and an increased necessity for glucose. It obligates cancer cells to consume bigger quantities of glucose to maintain a reduced state of metabolism, making cancer tissues very addictive to glucose.

ABA as Inhibitor of Alfa-Amylase

ABA is able in plants to reduce cations as K⁺ and Na⁺ from cytoplasm affecting its supplies of energy. This hormone is also able in plants to inhibit enzymes as alfa-amylase blocking hydrolysis of starch and interfering in supplies of glucose. Milborrow (1967) concluded that ABA might be the major component of inhibitor-beta. Likewise Hemberg et al (1961), pp. 861-867, showed that such inhibitor-beta identified by Milborrow (1967) as Abscisin II suppressed an activity of alfa-amylase, but only insignificantly affected an activity of beta-amylase in resting potato tubers. On 1967, Hemberg, p. 1666, confirms such conclusions. In addition, Manfield (1971), p. 147, suggested that a starch disappearance in guard cells occur simultaneously with K⁺ entry. Hence, during stomatal opening ABA is inactivated, not showing any effect on alfa-amylase. Likewise, during stomatal closure ABA inhibits alfa-amylase.

Na⁺ Mechanisms in Normal Cells

Researches of Cone (1974) clarified a relation between EPD also called electrical transmembrane potential (Eₘ) and associated ionic concentration differences in mitogenesis control of normal cells.

It has been pointed out that a cell multiplication or mitosis is stimulated by a depolarization of the cell. This phenomenon of potential fall is caused by increase of Na⁺ concentration in the cytoplasm.

According to Cone (1974), pp. 423-424, as a cells density increases, a substancial direct cell to cell surface contact begins to develop. Hence, a mitotic activity begins to decrease with a corresponding rise in membrane EPD.

Likewise, in order to get a quiescent stage, cells increase membrane EPD by decreasing Na⁺ concentration in cytoplasm.

In this invention, experiment of Cone (1974) has been interpreted by applying a derivation of the Nernst equation, as follows:
PD[mV]=61 log [Na⁺] e/[Na⁺]i.

where, EPD is measured in mV; [Na⁺] e is an extracellular Na⁺ concentration and [Na⁺] i is an intracellular Na⁺ concentration.

In such experiments of Cone only Na⁺ and K⁺ were measured, therefore and in agreement to Cone, these concentrations could not be directly related to the measured Eₘ in terms of the EPD. Nevertheless, one ion concentration can be related to EPD, if it is applied the already mentioned equation according to the concept of Nernst Potential. This concept considers only one type of ion in a cell, where there would not be any other ions, to know distribution of such an ion in that real cell, for a determined amount of potential.

That equation is derived from the Nernst potential equation as it follows:
V=RT/zFIn(Co/Ci)

where V=Nernst Poential in mV; R=universal gas constant (8.314 J/mol·K); T=absolute body temperature (321 K); z=charge on ion; F=Faraday constant (96485.309 C/mol); in or log of natural numbers=2.303 log₁₀; Co=concentration outside membrane; and Ci=concentration inside membrane.

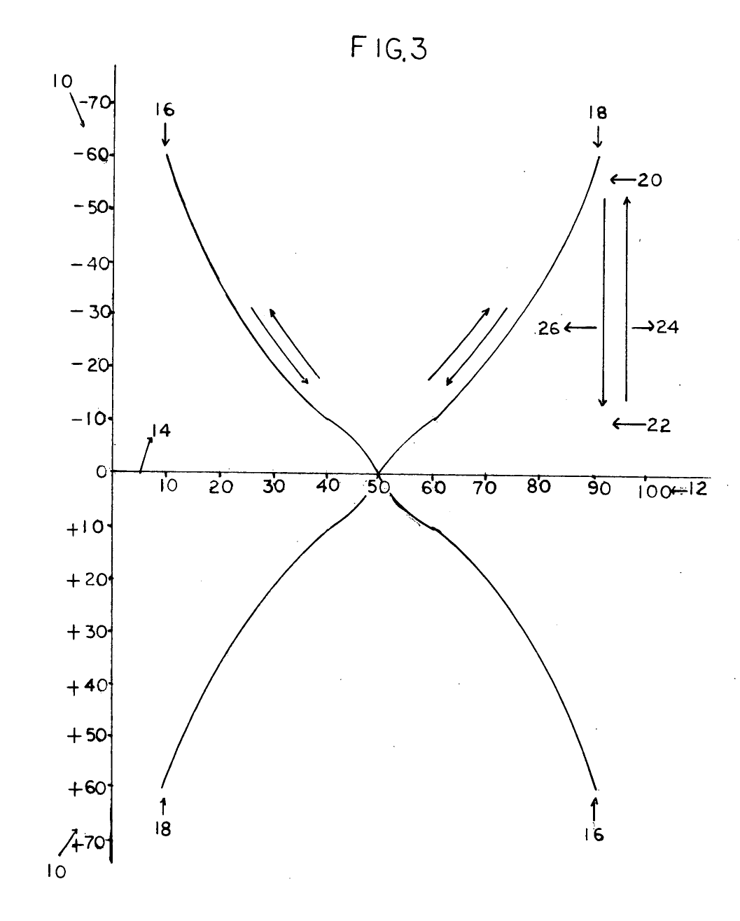

In FIG. 3 it is possible to observe a coordinate system, where y-axis is represented by the variable EPD (10) and x-axis is defined by Na⁺ concentrations in percents (12). X-axis divides the function in two planes. One upper plane is subject to negative values of EPD and a lower plane is subject to positive values according to convention. Quantitative results from using the equation are corresponding to a traditional coordinate system where the upper plane has positive values and the lower plane has negative values. Nevertheless, sign of values are changed by graphic convention of the scientific expression of the EPD concept.

Two curves define a double function. One curve is related to intracellular Na⁺ concentrations (16). The another one represents the extracellular Na⁺ concentrations (18). In the upper plane a depolarization is characterized by an increase in intracellular Na⁺ concentration (16) and a decrease in extracellular Na⁺ concentration (18), in the meantime EPD (10) gets lower values. Thus, the cell is able to pass from the quiescent stage (20) to the mitogenesis stage (22). Normally, cells get no further than the isoelectric point (14) and it can get back to the quiescent stage (20) by increasing extracellular Na⁺ concentration (18) and decreasing intracellular Na⁺ concentration (16).

Lower plane is defined by the same mentioned curves but obtaining both functions, EPD positive values (10). Cells can get this plane, with concentrations of intracellular Na⁺ (16) increasing over 50%, passing the isoelectric point as seen in FIG. 3. Thus, an intracellular positive charge inside of the cytoplasm obtains more positive values. Higher positive charges in cytoplasm can at certain point attract anions to the plasma membrane increasing a EPD in the lower plane or producing a stronger depolarization.

In Cone's Investigation (1974), pp. 423-425, it can be observed Na⁺ and K⁺ concentrations (mcmol/ml) in the mitogenesis stage (log phase) and the quiescent stage (saturated) for the used types of normal cells (CHO and 3T3) during the trials. For both types of cells, “a 50% of concentration of Na⁺ was decreased from the mitogenesis stage to the quiescent stage, and this same percent was increased. From the quiescent stage to the mitogenesis stage”. Values of EPD varied between −10 mV in mitogenesis stage and −65 mV in quiescent stage. Here also can be noted slight variations of K⁺ concentrations.

Experimental results by Cone (1974) are shown in the next table:

| | CHO MONOLAYER CELLS | | 3T3 MONOLAYER CELLS | |
|---|---|---|---|---|
| ION | MITOGENESIS QUIESCENT | | MITOGENESIS QUIESCENT | |
| Na⁺ | 15.3 +/− 1.8 | 7.9 +/− 2.1 | 17.6 +/− 1.5 | 8.6 +/− 0.8 |
| K⁺ | 186.1 +/− 5.3 | 185.9 +/− 6.2 | 204.5 +/− 3.6 | 197.0 +/− 4.8 |

This mentioned Na⁺ model, is correlated to the phenomenon of stomatal movements. Same physiological behavior explains shifts of stomatal closure and stomatal opening, according to variations of EPD. Hartung et al (1980) p. 260, pointed out that decreased intracellular K⁺ could generate membrane hyperpolarization of Lemma cells. Such hyperpolarization, means an increase in EPD in the upper plane that produces stomatal closure. Likewise, a depolarization of guard cells produces stomatal opening. A similar curve to FIG. 3 may delineate these stomatal mechanisms.

Here it is also evident that, cell hyperpolarization and decreasing intracellular Na⁺ induced by ABA, are effects conducting cells to get a quiescent stage. Proof of it is given, because ABA is able to produce dormancy and growth inhibition in plants (see reference of Dekock et al 1978, paragraph 0010). ABA mechanism of growth inhibition is associated to nucleic acids. On the other hand and in agreement to cone (1974) low Na⁺ level in cells is presumed to prevent synthesis of one or more mitogenically essential species of RNA. Likewise, a hyperpolarization by decreasing intracellular K⁺ in animal cells may be a mechanism induced by ABA too. Both models provide evidence that such elements (Na⁺ and K⁺) are correlative and operate a same and general mechanistic model with different effects, Na⁺ in connection to mitogenesis and growth inhibition, and K⁺ in connection to osmosis in plants as in animals.

Na⁺ Mechanisms in Cancer Cells.

Cone (1974), p. 431, remarked that a cancer cell multiplication or mitosis is characterized by A sustained and pronounced depolarization of cell in conjunction to an increase of Na⁺ concentration in the cytoplasm. This phenomenon of malignant proliferation blocks or negates the effective functioning of the ionic regulatory system resulting in a sustained depolarization of the cell with associated inability to lower the Na⁺ concentration to nonmitogenic levels. That inability of cancer cells to decrease Na⁺ would be associated to a reduction of the effective operation of Na⁺ pump.

Electrical changes as lower EPD in rapidly proliferating and transformed cells have also been reported by Binggeli et al (1986), in Breast Cancer by Marino et al (1994), and in Colon Cancer by Davis et al (1987) and Goller et al (1986).

In agreement to aforesaid statements, cancer cells are not able to switch to a quiescent stage, which provokes in such cells a perpetual and uncontrolled cellular proliferation.

sustained depolarization in cancer cells can be considered a deviated variant of the general mitogenesis model of normal cells as observed in FIG. 3. ABA action is capable to abort the malignant mechanism by shifting cancer cells from a depolarized and damage configurational state to a normal configurational state.

Modified Triple Layer Model

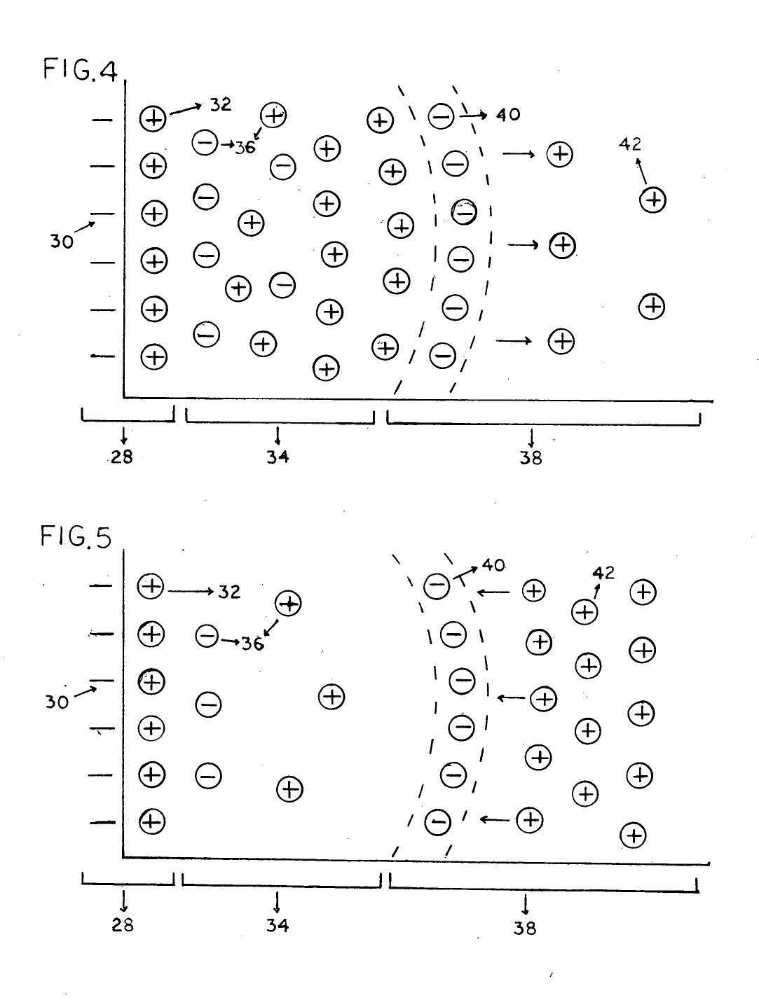

Membrane-associated HCG molecule of cancer cells configures theoretically according to this invention and FIGS. 4 and 5, a variation of the triple layer model. Three fundamental layers structure this model. They are:

1. A first layer (28), the innermost or surface layer, also called the Stern layer, which consists of the plasma membrane's solid surface itself with negative charges (30) and local positive ions (32).

2. A second layer (34) or diffuse layer defined by adsorbed negative and positive ions of relatively strongly bound (36).

3. A third layer (38) or HCG layer, consisting of negative charges of SIA (40) in the outer glycocalyx of HCG, plus adsorbed or repelled cations (42).

Many models have been developed which explain the behavior of a membrane. The diffuse double layer designed by Gouy (1910), the Stern model, the Gouy-Chapman-Stern-Grahame electric double layer and the triple layer model have widely been studied.

Original triple layer model in normal cells consists of a diffuse third layer with ions weakly attracted to the solid surface. This triple layer model in this invention is modified due to the presence of HCG in cancer cells, which makes tumor cells behave in an atypical fashion. It functions in cancer cells, as an electrical barrier. There, negative charges (40) exert the important role of repelling cells of the immune system and serve also as receptors.

Some researchers have mentioned that absorption of substances by the plasma membrane can be considered an electrostatic mechanism where diffusing mechanisms are also involved. An oppening of Na⁺ channel will cause absorption of that cation, which will establish concentration gradients in the direction to the cytoplasm.

Low Na⁺ concentrations in first and second layers (28) and (34) are able to produce a concentration gradient between the third layer (38) and these inner layers (FIG. 5). Thus, Na⁺ can be diffused to inner layers and subsequently to the cytoplasm.

Cations as Tools for Changing Polarization of HCG

According to Van Slyke (1933), p. 184 from one third to three-fourths of the mineral base in the cellular cytoplasm in normal cells must be neutralized by complex acids, chiefly the proteins and the phosphatides, which are alike in being buffers and indiffusible colloids. Bases such as K⁺ and Na⁺ are anchored in the cells in which they form components.

These mentioned cations can be involved in binding to SIA. Tiralongo (2002), p. 4, mentions in the apart of biological roles of SIA that, due to negative charges, SIA are involved in binding and transport of positively charged molecules, as e.g. Ca²⁺. Nevertheless, according to Delong et al, 1950 p. 718 and Ambrose et al, 1956 p. 576 cellular surface of cancer tissues show a decreased Ca²⁺ content in comparison with normal tissue. Deficiency of Ca²⁺ has been associated to linking decreased adhesiveness and invasiveness of cancer cells.

Cations such as Ca²⁺, Mg²⁺ and others are not able to neutralize HCG. Instead, cations such as Na⁺ and K⁺ may be considered physiological tools for neutralizing negative charges of HCG because a transport of such cations exerts drastic changes in EPD. ABA can be medically used in humans as an ion exporter for moving these cations to the outside of plasma membrane. Concentration gradients reversed by Na⁺ toward the extracellular fluid (ECF) have been found in relation to a cancer patient curative process. Direction of the diffusion flux is defined by higher concentrations of Na⁺ contained in first and second layers (28) and (34) and lower concentrations of cations contained in third layer (38), (FIG. 4).

According to clinical observations of Dr. Max Gerson when cancer patients were responding to treatment they lost extra Na⁺ from the body in the urine (cited in Cope F. W (1978), p. 466). These observations of Dr. Gerson are clearly indicative that, Na⁺ is eliminated from cancer cells during the process of recovery and K⁺ is not extruded.

According to Dr. Gerson the other part of the process of recovery of the human body from cancer disease was replacement of excess Na⁺ by K⁺ in damaged tissues. Those results obtained and developed by Dr. Gerson during 30 years of clinical experimentation are found correlated to thesis of Ling (1960) and Cope (1978) about the association-induction hypothesis and tissue damage syndrome (see paragraph 0064). Extruded Na⁺ and regained K⁺ are effects or consequences of a change of configuration state in the cancer cell, in which the malignant cell switches from a damage configuration state to a normal state. Thus, sudden bioelectrical changes take place in negatives charges of intracellular protein matrix. There, after malignant mechanism is aborted proteins lose preference for Na⁺ and regain preference for K⁺.

Healthy cell is associated with higher intracellular K⁺, lower intracellular Na⁺ and higher EPD, and cancer cell is associated with lower intracellular K⁺, higher intracellular Na⁺ and lower EPD (Cone 1975, cited in Haltiwanger 2003, p. 30). At this point, it is possible to predict that membrane potential (EPD) is the essential factor for switching from a damage configurational state to a normal state. A variation (increase) in EPD inexorably conducts in cancer cell toward a normal configurational state.

ABA exporting both cations outside of cell provokes a membrane hyperpolarization which aborts mentioned profound and sustained depolarization of cancer cells. High mobility of K⁺ and acquired normal configuration state of negative charges in proteins will facilitate that K⁺ can be regained by the cytoplasm. Low mobility of Na⁺ and a normal condition of the cell after hyperpolarization will produce a healthy cellular metabolism where this element is not longer required in quantities regularly absorbed by cancer cells.

In agreement to Haltiwanger 2003, p. 41, some effects that are seen when membrane potential (EPD) is increased include: enhanced cellular energy production (ATP), increased oxygen uptake, changes in entry of Ca²⁺, “movement of Na⁺ out of the cell, movement of K⁺ into the cell”, changes in enzyme and biochemical activity, and changes in cellular pH.

Thus, wether K⁺ is absorbed back to the cell and Na⁺ is extruded after the damage configuration state has been changed, it is evident that cations are not coupled to SIA negative charges of HCG outer layer. This HCG phenomenon of “no coupling” to positive ions after a cancer cell is hyperpolarized is due to that, in addition and simultaneously, a “positive polarization effect” might occur in the HCG outer layer repulsing Na⁺ to the extracellular fluid.

It is believed in this invention that, positive polarization of HCG outer layer may be produced through a removal of electrons from HCG outer layer, toward cations located in first and second layers (28) and (34), after such cations are massively exported to outside plasma membrane.

This last statement denotes that a subtle electrostatic phenomenon is exerted involving electric interactions outside the plasma membrane after ABA hormonal action.

Electrostatic Phenomenon in Plasma Membrane

According to a large number of researchers, total cell structure is connected through a liquid crystal protein polymer connective system continuum. This term has been used to express that such system connects the cytoskeletal elements of the inside through cell membrane, as a total structure. Haltiwanger 2003, p. 20, reveals that such a continuum of liquid crystal connections in cells, deeply studied by Becker (1974), Ho MW (1998) and Oschman J L (2000) allow electrons and photons to move in and out of cells. In his opinion “cytoskeletal filaments” function as electronic semiconductors and fiberoptic cables integrating information flow both within the cell and with other cells. Also it is believed that cytoskeleton proteins, link the inside of cell like a system of telegraph wires terminating onto the nucleus membrane (Ho 1993, p. 94) or act as coherent molecular antennas radiating and receiving signals (Oschman 2000, p. 131).

Dr. Merrill Ganett (1998), has studied for decades the role of charge transfer and electrical current flow in the cell. He believes that DNA, cytoskeletal proteins and cell membranes transmit an inward and outward current. The inward current flows from the cell membrane to cell structures like mitochondria and DNA and the outward current flows back along liquid crystal semiconducting cytoskeletal proteins through the cell membrane to the extracellular matrix.

Nature of cellular electron movement in transport systems as it ocurrs for example in mitochondria and chloroplasts has been well known, but outside transport systems is poorly understood (Stern et al 1999, p. 368). Certain theories has been proposed. Through a total cell, electrical transmission must flow on protein surfaces. According to Adey (1988), p. 149 Electrical interactions between cell membrane and weak electromagnetic fields are exerted through electrical charges located on cell surface macromolecules. Proteins and macromolecules function as semiconductors (Szent-Gyorgyi A 1978,85, Brillouin L 1966). In addition to semiconductivity, complex crystalline structures possess properties such as photoconductivity and piezoelectricity (Becker 1999, p. 237). In proteins, at a specific level, a passage of electrons is produced through a major cytoskeletal component (actin) by which ionic currents are induced. Cytoskeletal structures can behave as electrical wires and are capable of functioning as nonlinear inhomogeneous transmission lines (Lin E et al 1993, cited in Stern et al 1999). Concentric and structured water surrounding proteins must also interact as dipole conducting electrical currents.

These filaments inside and outside of cells can be the vehicles of electrical currents when negative charges of proteins get in contact with cations. The electroscope is a device, which transmits electrical currents such as it could occur through protein negative surface charges of mentioned filaments in cells and structured water.

The Electroscope Device

Haltiwanger (2003) p. 6, has cited that there are multiple structures in the cell acting as electronic components due to that biological tissue and components can receive, transduce and transmit electric, acoustic, magnetic, mechanical and thermal vibrations. For example, membrane proteins and DNA consist of helical coils. These structures function as electrical inductors (Haltiwanger 2003, p. 5). Cell membrane functions as a capacitor with the characteristics of a leaky dielectric (Garrison W, 1969).

Novel prize Szent-Gyorgi a resembled cell membranes as closely analogous to the PN junction, a semiconductor device used in solar cells, which facilitates the separation of positive and negative charges, and is capable of generating an electric current when excited by heat or light (Ho 1993, p. 102).

the electroscope device, which determines or measures presence of electrostatic forces has a strong correlation to the theory of electron transfer in liquid crystal continuum in cells through filaments in and out of the cell. In graphic 6 and 7 of this invention can be observed two identical electroscopes in contact with negative and positively charged bodies. Different parts or elements of the electroscope have a similar element in the plasma membrane of normal and cancer cells, according to the following similes: the metal ball (44) can be considered receptors of SIA with negative electric charges in the plasma membrane outer layer. The metal rod (46) can be considered glycoprotein projections coming from the membrane-associated HCG molecule. The metal leaves (48) can be considered the plasma membrane and Na⁺ channels. The negative and positive charged bodies of the elctroscope can be considered ions as for example Na⁺ (54). The electron (56) and the electron transfer (58) have no simile, because they are naturally identical. The glass container (52) and gasket of the electroscope (50) can be considered the isolating properties of the lipid bilayer of plasma membrane.

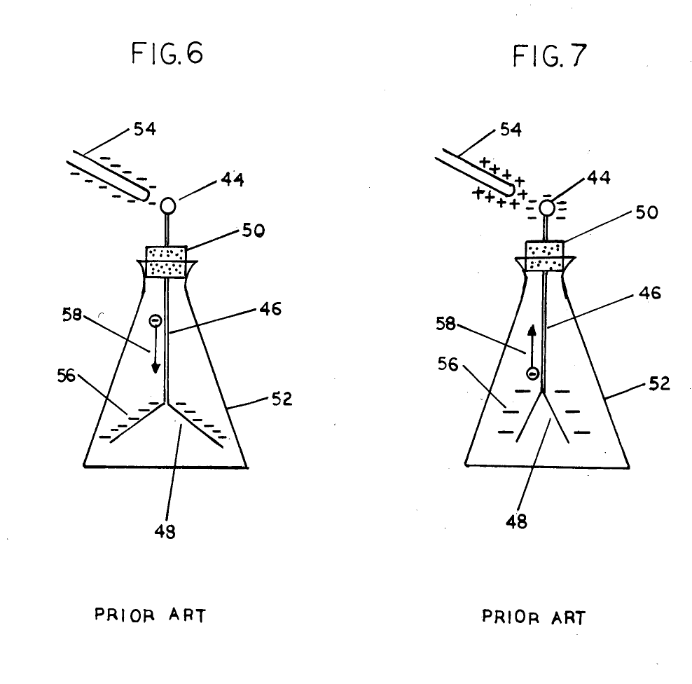

The elctroscope permits to understand that a transference of electrons may be given from the plasma membrane to receptors of SIA in outer layer. In FIG. 6, if it is approached to the metal ball (44), a negatively charged body (54), for example a frictionally resin rod, some electrons (56) are transferred (58) through the metal rod (46) toward the extreme of the device where the metal leaves (48) are located. Such metal leaves (48) will widely open due to coulombic forces, which repel charges of same polarity. In FIG. 7, if it is approached to said metal ball (44), a positively charged body (54), said metal leaves (48) get closer, due to that electrons (56) are transferred (58) to the metal ball (44). Likewise if this metal ball (44) comes in contact with the ground, for example touching it with a finger, some electrons (56) will escape from the glass container of the electroscope (52). Electric interactions in cells such as depolarization may be explained through the electroscope principle.

Depolarization of Plasma Membrane Caused by Ionic Currents

A model developed by Tsong (1989) has postulated that a protein can undergo conformational changes by a coulombic interaction with an oscillating electric field.

It is possible to assume that cation attraction to negative charges of sialoglycoproteins in the plasma membrane and proteins of ion channels can generate electrical changes in such proteins.

According to the elctroscope principle, electron transfer may occur from the plasma membrane to currents of cations causing a removal of negative charges in the plasma membrane and ion channels. When said electrons are removed, conformational changes happen in the plasma membrane, most importantly a depolarization.

According to Bennett et al. (1997) in an article titled “Contribution of sialic acid to the voltage dependence of sodium channel gating. A possible electrostatic mechanism”, changes in Na⁺ ion channel of rat skeletal muscle were observed after enzymatic (neuramidase) removal of sialic acid. Such changes were 10 mV more depolarized than control channels. Bennett et al. also point out that a general future of many sodium ion channels is that they are heavily glycosylated, with a large fraction of carbohydrate in the form of SIA. SIA negative charges removed from surfaces of ion channels is found associated to depolarization.

Electrostatic Phenomenon Hypothesis in Na⁺ Channel.

In aforesaid statements it can be observed that an interaction between currents of cations and SIA negative charges of Na⁺ channels, may be given through an electrostatic phenomenon.

From a general knowledge, a depolarization effect in the plasma membrane produced probably by removal of electrons (negative charges) has a consequence in opening the channel.

opening and closing ion channels can be interpreted in this invention through the electroscope principle, to demonstrate that electron transfer is the mechanism underlaying in ionic interactions in the plasma memmbrane.

According to Marban et al (1998), p. 647, Na⁺ channels consist of various subunits, but only the principal (alfa) is required for function. The alfa subunit has a modular architecture: it consists of four internally homologous domains (labeled I-IV), each of which contains six transmembrane segments. The four domains fold together so as to create a central pore. According to Stuhmer et al (1989), the fourth transmembrane segment (S4) stereotypically studded with positively charged residues, lies within the membrane field and moves in response to depolarization, somehow opening the channel. In agreement to Bennet et al (1997), p. 327 the segment (S4) Consists of a repeated triad of two hydrophobic amino acids followed by a positively charged residue, consistent with a role as a voltage sensor, residing within the membrane bilayer. The rest of the segments of the Na⁺ channel are negatively charged with SIA. Schematic depictions of the Na⁺ channel alfa subunit are shown by Marban et al (1998), p. 648.

According to Catterall, W. A (1992) it is assumed a helix model where each positively charged residue in the (S4) segment is paired with some negative charge on the adjacent S1 to S6 segments (cited in Aidley et al 1996, p. 186). This model appropriately suggests that coulombic interactions (attraction and repulsion forces) occur between paired negative and positive charges in channels.

On the other hand, Hodgkin and Huxley (1952) found that changes in ionic permeability were associated with the movement of some electrically charged particles within the membrane. Thus, movement of charged particles may be given toward positive charges of segment (S4) or from negative charges of adjacent segments of channels.

Stuhmer et al (1989) also found that the steepness of the relation between channel opening and the membrane potential was progressively reduced as the positively charged residues of the (S4) segment were replaced by neutral or negatively charged residues. This research automatically realizes that, movement of charged particles mentioned by Hodgkin and Huxley (1952) happens in negative charges of adjacent segments of channels.

In this invention it is disclosed under a viewpoint of the elctroscope principle that, currents of cations close to the channel will produce a removal of electrons from the negatively charged segments of the channel, decreasing negative charges of such segments or increasing positive charges. When it occurs, segment (S4) is moved away from the rest of the segments due to coulombic forces of repulsion. At that point, same polarity as (+) it characterizes to all the segments. Electrons removed from the channel, before a cation is entered, neutralize said cation or current of cations. Thus, they pass from an ionic stage to a neutral or uncharged stage. Therefore, when cations get through the channel, negatives charges acquired before they enter to the pore are incorporated back to the channel. After the process is executed and once electrons get back to adjacent segments during the passage, segment (S4) with positive charges become it attracted to the rest of segments, which they are again negatively charged. When the cation exits from the channel, it becomes to its original ionic stage due to electron transfer to the channel. So, charges of the ion channel and exited cation turn back to their original electrochemical condition. It means that an electrostatic balance is kept in plasma membrane and channels after the transport is executed, and electric transmembrane potential difference is finally defined by elements in its ionic stage after passage of cations is carried out.

The lipid structure of a cell membrane makes it relatively impermeable to the passage of charged molecules. This property can become it in a cell membrane general rule, if charged molecules get through ion channel as uncharged molecules according to gating theoretical model expressed in this invention.

HCG Positive Polarization by ABA

A decreasing of cations from cytoplasm to the outside of cell will produce electrical or electronic changes in the membrane glycocalix interface (38) conducting it to lost of electrons which they carry negative charges.

Removal of electrons from HCG outer layer is increased when cations as Na⁺ and K⁺ are exported outside of cell in a compartmental area close to plasma membrane in layers (28) and (34) of the modified triple layer model.

According to the electroscope device, if positively charged bodies or cations get close to said metal leaves (48), electrons (56) will be transmitted from the metal ball (44) to the metal leaves (48), winning it negative charges the metal leaves (48) and surrounding bodies. Likewise, the metal ball (44) loses negative charges. In this invention the simile of metal leaves (48) are plasma membrane and Na⁺ channels, and the simile of metal ball (44) are the receptors of SIA in HCG outer layer.

this principle of the electroscope can be applied to a cell due to that it displays electrical properties.

it mechanism will be severe to cancer cells, due to that ABA is able to extrude cations as Na⁺ or K⁺ from cytoplasm to locations outside plasma membrane. It brings as main consequences: lost of electrons in SIA receptors expressed in outer layers as first stage of the curative cancer process referred by Dr. Gerson and Na⁺ concentration gradients reversed as final stage.

Aforesaid statements in this invention address Dr. Livingston theory about properties of ABA of neutralizing HCG and researches of Tanada (1971) and Hartung et al (1980), about properties of ABA of producing positive polarization and hyperpolarization effect in plasma membrane.

ABA Signalling Hormonal Pathway and Others Multiple Effects

Four reasons provide secure evidence that, ABA can generate in animals, the same effect produced in plants:

1. Identical signalling pathway controlled by G-proteins in activation of ion channel in relation to K⁺ has been characterized, in plants as in animals.

2. K⁺ is found in normal cells of plants and animals, as the principal cation inside of cytoplasm. ABA has influence over it cation.

3. Na⁺ is the principal cation inside of animal cancer cells and ABA also has influence over it cation.

4. It has been proved that another group of plant hormones as cytoquinins produce effects in humans.

A change in the behavior of the plasma membrane in cancer cells may cause a shift, directing such cell to a hyperpolarization condition. That mentioned change can be caused by an ABA signal. When the hormone acting as first chemical messenger in the circulatory system gets in contact with a receptor out face plasma membrane, it gives a signal through a second chemical messenger to decrease K⁺ and/or Na⁺ from cytoplasm. The intracellular messenger and extracellular ligand for G-proteins related to ABA has been identified as sphingosine-1-phosphate (SIP), which is produced by the enzyme sphingosine-kinase (see paragraph 0031). According to Kennedy B-K (2003), from Penn State University, plants respond to environmental stresses with a sequence of molecular signals known in humans and other mammals as the G-protein signalling pathway. In human cells this mechanism has been recognized as responsible in regulating either the opening of ion channels and activities of intracellular enzymes. Kennedy also mentions that a large percent of drugs approved for use in humans target the G-proteins signalling pathway and that these findings could be used in the search of leading to new drugs for human diseases.

In addition to this HCG neutralizing effect of ABA, this hormone may also be able in plants to produce others multiple effects: 1. A lysiginous breakdown of membrane cells. 2. Inhibition of protein synthesis and nucleic acids. 3. Inhibition of alfa amylase, blocking glucose availability and transport. 4. Reduction of water potential by increasing hydraulic conductivity of plasma membrane. 5. Alteration of Na⁺—K⁺ ion pump mechanisms. 6. Promotion of aging and senescence of cancer cells. 7. Promotion of dormancy and growth inhibition.

Preparation of the Medication

Curve of Uptake Efficiency of ABA

ABA behavior of an intra and extracellular pH in plant cells will be taken as reference for choosing the right pH for a buffer solution. It will permit to figure out consequentially a pH of the medication to get the best treatment of ABA against human cancer cells. This medicine has not been applied before in humans by intavenous way, therefore ABA behavior in plants is the only available reference.

An efficient response of ABA in plants can be evaluated by its capacity to inhibit stomatal opening in certain conditions. ABA concentration applied via extracellular and pH of the medium are important factors. Anderson (1994), p. 1177, found that extracellular aplication of 10 mcM ABA, Inhibited Stomatal Opening by 98% at pH 6.15 and by 57% at pH 8.0. In this same research he also mentioned that, this pH dependence of extracellular ABA action might suggest a contribution of an intracellular ABA receptor to stomatal regulation.

In agreement to Allan et al 1994, p. 1107, ABA on guard cells is more effective at pH 5.5, than at pH 7.

Efficiency of ABA uptake in this invention must be measured as function of: stomatal closure, pH of the extracellular medium, and receptors outside of plasma membrane. Absorbed quantities of ABA are not directly proportional to stomatal closure.

A receptor site of ABA, still remains unknown. In spite of the fact, Hornberg et al (1984), pp. 321-323, found ocurrence of a high-affinity guard cell specific ABA-binding proteins facing the apoplasmic space. They detected three different designated sites: as anion (one site) and for ABA in its protonated form (two sites).

Microinjections of ABA into guard cells did not inhibit stomatal opening according to Anderson (1994), p. 1182, and, Popova (2000), p. 379. Such data provides evidence that a reception site for ABA is on an extracellular side of the plasma membrane, by which it also suggests that a presence of ABA in the apoplast is necessary for inducing stomatal closure. Nevertheless, research of Pedron et al (1998), p. 390 agrees with a dual location of ABA reception sites (intracellular and out-facing plasma membrane).

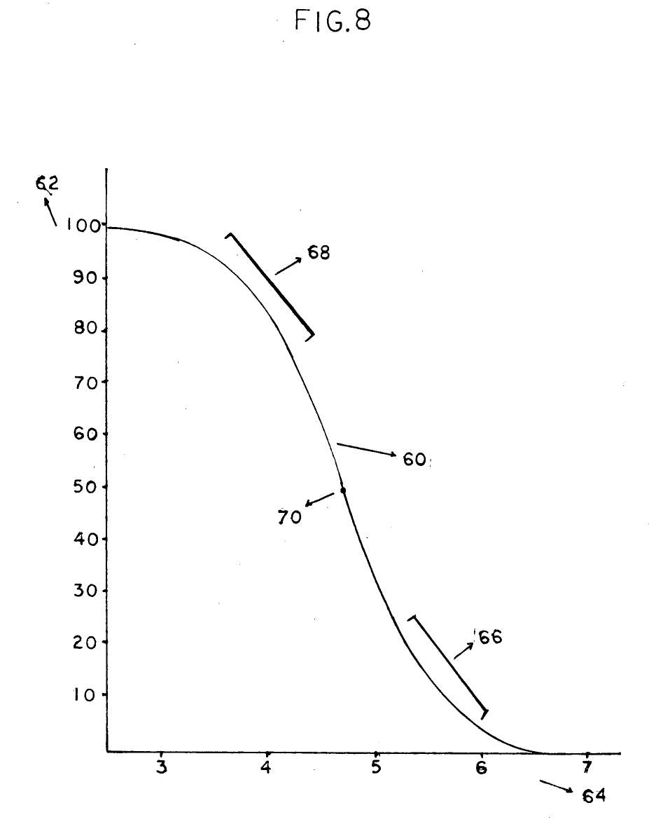

A theoretical curve of ABA uptake (60) measured in percent of concentrations (62) In relation to values of pH outside the plasma membrane (64) has been drawn in the FIG. 8, according to the Henderson-Hasselbalch equation:
pH=pK+log [conjugate base]/[conjugate acid]

As the protonated or undissociated form of ABA is ABAH, and the anion is ABA⁻, the mentioned equation results as:
pH=pK+log [ABA⁻]/[ABAH]

In the equation ABA pK has a value of 4.7 (70).

values of pH (64) were calculated by considering relative proportions in percent (62) for concentrations of the two different forms of ABA.

As it can be observed in the FIG. 8 (ABAH) uptake concentrations (62) increase when it decreases values of pH (64), by that way ABAH can get through plasma membrane. According to the FIG. 8 A 30% of ABAH uptake is enough to produce maximum efficiency of stomatal closure (66), between a range of pH of 5 and 6, whether it is considered above information of experimental data. Likewise, it is possible to define on the curve an area of minimum efficiency (68) with an ABA uptake over 70%. At this point, ABA absorption has been completed to initiate stomatal opening. The whole of this process will depend: on pH outside cytoplasm and water deficit.

This area of maximum efficiency (66) and pH will be taken in consideration to manufacture a buffer solution for the medication.

Concentrations and Dosage for Medication

In survey of Hartwell 1982, an enormous quantity of plant species has been found with properties against different types of cancer generating such species perceptible benefits. In plants, ABA concentration is considered relatively very low; notwithstanding ABA is the commom factor found in all species of plants. ABA concentrations in unstressed leaves range approximately about 800-1500 ng/100 g of fresh weight while, in water stressed leaves ABA ranges about 1700-10000 ng/100 g of fresh weight (Singh et al 1979, p. 136). ABA levels increased due to water deficit by at least in an average of 2-7 fold.

ABA concentrations in animals as pigs ranged between 13-180 ng/100 g of fresh weight of tissue in different organs. In rats fed with diet containing ABA, concentration was determined between 248-429 ng/100 g of fresh weight of brain tissue (Le Page-Degivry 1986, p. 1156). Concentration of ABA in animals is lower than is found in plants.

In this invention, concentrations of ABA are taken from Dr Livingston's experiments in vivo. ABA concentrations used by Dr Livingston are higher than ABA concentrations in plants as seen paragraphing [0160]. The most efficient group showed in the experiment was the number II, with 90% of survivors and a lower dose (1 mg/kg). Such concentration can be taken as a reference and defined as equivalent to 1 mg/ml. Nevertheless doses can range between 0.1 mg/kg and 20 mg/kg, and concentrations between 0.1 mg/ml and 50 mg/ml. Doses may vary according to a system of application.

ABA effect can be reproduced once again by repetitive doses, which is necessary because ABA provokes a transient effect. ABA is involved in homeostatic mechanisms in plants by which repetitive doses can produce a prolonged effect. Hence, a continuous positive polarization of surface charge will permit to immune system cells get close to cancer cells and destroying them.

Preparation of Buffer Solution

During the first stages of cancer a pH of the bulk (blood) oscillates around 7 (84), but because of cancer progression this pH can get lower values of around 6.5 or less. Cancer cells have mechanisms to adjust pH surface responding to changes of pH of the bulk. As e.g. They can keep acidity in vesicles inside of cytoplasm or absorb Na⁺ by exchange with H⁺ to reduce pH.

An ABA medication is buffered at a pH 6.1 according to FIG. 8 due to that such pH correspond to the area of most uptake efficiency in stomatal closure in plants (66). Buffer pH of the medication becomes equal to the pH of the bulk in this case, because the buffer solution containing ABA will hold a pH of the blood until ABA is absorbed or as long as buffer capacity is permisible. Thus such pH is applied, being favored a better ABA uptake.

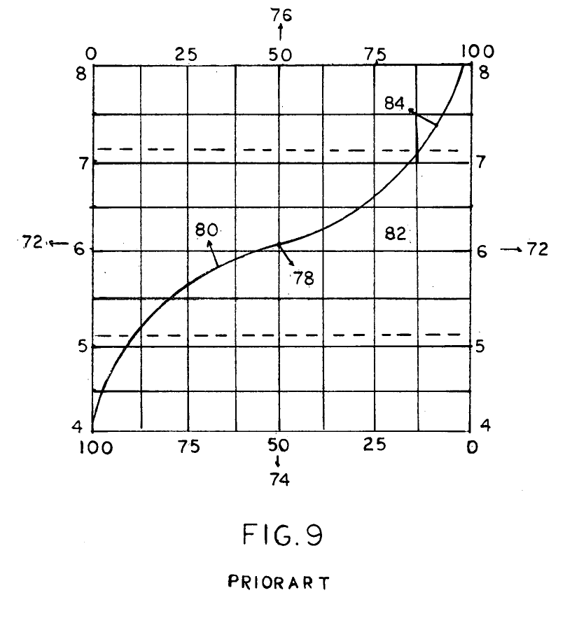

A buffer solution for the medication is prepared using the method of Cassiday, (1999), pp. 10-11. For making the solution, it has been elected carbonic acid and its salt (H₂CO₃—HCO₃⁻), which has a pK=6.1 (78). Graphic of the curve (80) has been showed in this invention in FIG. 9 as previous art. Curve is elaborated by the author applying the Henderson-Hasselbalch equation and plotting values of pH between 4 and 7 (72) and relative concentrations in percents of carbonic acid (74) and bicarbonate (76).

Buffer solution is fabricated using 50% of carbonic acid and 50% of its salt. As pH=pK, buffer solution will have an optimal capacity inside of the region of maximum buffering capacity (82). Due to ABA is a weak acid it will lightly modify the buffer solution once it is prepared. Thus, salt will lightly increase and carbonic acid decreases.

ABA Isolation, Production, Marketing Forms and Handling

Isolation of ABA was defined by Addicott et al (1969), p. 141, as it follows:

1. Extraction with an aqueous organic solvent.

2. Acid-base fractionation to yield organics acids.

3. Gradient elution of carbon and/or silicic acid column chromatograms.

4. Paper and/or thin-layer chromatography followed by crystallization from the eluate.

Production of ABA by using the fungus Cercospora rosicola as mentioned by Assante et al (1977), pp. 1556-1557, can carry out a comercial level. A strain denominated Cercospora rosicola Passerini, frequently found on Rosa sp, produces 6 mg/100 ml maximum of ABA. It must be cultivated on a potato-agar medium, at pH 6.5-6.8, under 24 degrees centigrade in the light for 30-40 days.

ABA production obtained from C. rosicola is considered high, wether it is compared with ABA yields from plant materials. Addicott et al (1969), p. 142, showed that such yields of plants ranged between 7 and 40 mcg/kg. A higher yield of 9 mg was obtained by processing 225 kg of dry weight from Gossypium fruit which yielded 40 mcg/kg.

From Sigma-Aldrich can different forms of ABA (cas number 21293-29-8, molecular weight 264.32) be purchased, such as: (+) abscisic acid 99% purity as the natural ocurring; (+/−) cis, trans-abscisic acid-3H (G) 95% purity as synthetic substance; (+/−) abscisic acid 98-99% purity as synthetic substance; (+) abscisic acid 98% purity as natural isomer; (−) cis,trans-abscisic acid as racemic or enantiomer.

Pharmaceutical material must be handled under protection from light and storage temperature of 20 degrees centigrade.

### EMBODIMENT NUMBER ONE

A pharmaceutical medicine for an intravenous, intramuscular and subcutaneous treatment may be elaborated by using the buffer system as mentioned before. ABA concentrations can range between 0.1 mg/ml and 5 mg/ml. Medication will be prepared by obtaining an isotonic solution not higher than 9 mg/ml (0.9% W/V). Volume to be applied will depend on doses and patient weight. Doses range between 0.1 mg/kg and 5 mg/kg.

### EMBODIMENT NUMBER TWO

Le Page-Degivry et al (1986), demonstrated in the article Presence of abscisic acid, a phytohormone in the mammalian brain, that ABA as molecule keeps its structure and properties after it is consumed. In such experiment, rats were fed with a diet containing ABA. Determination of the hormone in tissue by radio immunoassay, after experiment, detected concentrations of ABA between 248 to 429 ng/100 g of fresh weight tissue.

Dr. Livingston's experiment using mice also proved that, a use of ABA by oral via had effectivity killing myeloid leukemia. Nevertheless, better results during the Dr. Livingston's experiment were obtained with a dose at 100 mg/kg (90% of survival) than a dose at 10 mg/kg (60% of survival).

Concentrations used to manufacture capsules can range between 10 mg/kg and 100 mg/kg.

Active principle of ABA can be prepared by using an acceptable carrier as a vehicle and packing such principle in capsules with a biodegradable dark coating to avoid isomeric changes due to the light.

### EMBODIMENT NUMBER THREE

ABA by catheter induction to a determined area of a tumor for inducing senescence can be considered a viable method to control terminal types of cancer. Nevertheless, it may result a more expensive treatment. Causing senescence against cancer this treatment brings itself use of a massive dose of ABA. Hormone concentration in the medication at 50 mg/ml, which is diluted in ethanol and administered without buffer by using a catheter will permit a fluid passage of the hormone to be directly applied to a tumor.

A (+/−)-abscisic acid manufactured by Sigma-Aldrich is able to carry the mentioned concentration in ethanol which may be clear to slightly hazy. Such synthetic form of ABA (cas number 14375-45-2) is obtained through a plant cell culture tested with 99% of purity.

Side Effects

Dr. Livingston reported during experimental observation, that ABA apparently had no toxic side effects in mice even when administered (i.p) in amounts up to 10% by weight of mice. Thus, considering 28 g body weight mouse and ABA (i.p) administered at 2800 mg per week, ABA had no adverse side effects. Nevertheless, ABA can not be prescribed during pregnancy due to existence of HCG in placenta. ABA being able of neutralizing that hormone might cause it abortion.

Operation

An administration for boosting the immune system with any natural or pharmaceutical medication during ABA treatment is recommended for pressing the immune system cells to attack cancer cells. Radiation or chemotherapy administered before, during or after ABA treatment is not recommended in this invention, because those traditional treatments weaken the patient's immune system. A simultaneous and coordinated action adopted from a patient and physician strengthening the immune system and neutralizing hormone of cancer will be an indicated treatment against cancer under the specifications of this invention.

Destruction of cancer tissues by any treatment it also brings to a process of released toxins, which can poison other tissues conducting to coma and death. Therefore, it is recommended in a patient recovery processs a Dr. Gerson method for detoxifying the human organism during ABA treatment. Also it is advisable to start an ABA treatment when large tumors has been removed by surgical procedures.

### CONCLUSIONS

Testimony declared by Dr. Livingston thirty years ago, in reference to ABA capacity for neutralizing HCG has been confirmed. In fact, a positive polarization of HCG is produced with a change in the configurational state of cancer cells. Clearness about existing compatibility between ABA as medicine to fight cancer and the disease is evident and can be perceived through the invention. Any medicine proposed for cancer must come into the conjunction of the recovery process mechanism of Dr. Gerson. Apparently the electron transfer in cancer cell membrane can be considered a missing piece of the puzzle for understanding the phenomenon of a cancer patient recovery process. Ideas, theories and references of the invention can be used as tools for better understanding the cancer disease, so as expressed in general terms by Ho I N 1993:

“As in any attempt to understand, we use whatever tools we have at our disposal to help us think, and good scientific theories are just that a superior kind of tools for thought”.

It will be apparent to those skilled in the art that modifications can be made without departing from the object and scope of the present invention. Therefore, it is intended that the invention only be limited by the claims.

What is claimed is:

1. A method of medical treatment against all types of cancer by using a medication with any of a natural and synthetic form of abscisic acid, related and derivatives, comprising:
(a) an oral medication for patients in its preliminary stage of the disease, and
(b) an intravenous/intramuscular/subcutaneous treatment for a middle stage of the disease, and
(c) an administration of the medication by catheter induction to a determined primary tumor for inducing senescence in terminal types of cancer, and

2. The method of claim 1, wherein said oral medication is administered in capsules.

3. The method of claim 2, further including an acceptable pharmaceutical carrier (a thickening agent) as vehicle for said capsules.

4. The method of claim 2, further including capsules fabricated with a dark coating to prevent isomeric changes of the abscisic acid by light.

5. The method of claim 2, further including for oral via, abscisic acid concentrations ranging between 1 mg/gr and 100 mg/gr, and doses ranging between 10 mg/kg and 100 mg/kg body weight.

6. The method of claim 1, wherein said intravenous/intramuscular/subcutaneous treatment is applied with a liquid medication systemically administered through infuses or injections.

7. The method of claim 6, further including in said liquid medication a pharmaceutical carrier solution containing as solvents an alcohol as first solvent denominated ethanol ranging it between 1 and 5%, and distilled water as second diluent to complete a volume of the medication.

8. The method of claim 6, further including an addition of a buffer solution to the medication.

9. The method of claim 8, wherein an election of a pH for said buffer solution is made according to a curve of the abscisic acid uptake showed in FIG. 6, wherein a pH equal to 6.1 is elected.

10. The method of claim 9, further including for said buffer solution carbonic acid (50% of a concentration) and bicarbonate (50% of said concentration), buffered at a pH 6.1, according to FIG. 7.

11. The method of claim 6, further including for intravenous/intramuscular/subcutaneous way, abscisic acid concentrations ranging between 0.1 mg/ml and 5 mg/ml, and doses ranging between 0.1 mg/kg and 5 mg/kg.

12. The method of claim 6, further including that a total concentration of said liquid medication will not be higher than 0.9% (9 mg/ml), to be used and prepared as an isotonic solution to equal blood concentration.

13. The method of claim 1, wherein the medication for catheter induction is administered with infuses to a determined primary tumor.

14. The method of claim 13, further including an administration by catheter induction by using abscisic acid concentrations ranging between 10 mg/ml and 50 mg/ml, and doses ranging between 0.1 mg/kg and 5 mg/kg body weight.

15. The method of claim 1, wherein a mechanism of action is based in a sodium and potassium extrusion caused by abscisic acid in cancer cells, comprising:
(a) changes in the bio-electric properties of cancer membrane cells and negatives charges of intracellular protein matrix of said cells, and
(b) said changes are provoked by a shift, directing cancer cells to a hyperpolarization condition according to FIG. 3, and
(c) an increase of membrane potential difference such as said hyperpolarization brings as final result a conversion of a cancer cell expressing a damage configuration state, getting it back to a normal state.
(d) another final result of the increase of membrane potential is a positive polarization of a cancer cell human chorionic gonadotropin through a removal of electrons, which it attracts human immune system cells to malignant tissues and permits to said cells to destroy such tissues.
